(19) United States
(12) Patent Application Publication
(10) Pub. No.: US 2021/0298772 A1
(43) Pub. Date: Sep. 30, 2021

(54) MICROCATHETER GUIDEWIRE UNIT, ROBOTIC CATHETER SYSTEM, AND MEDICAL SYSTEM

(71) Applicant: Siemens Healthcare GmbH, Erlangen (DE)

(72) Inventors: Christian Kaethner, Forchheim (DE); Andreas Meyer, Bubenreuth (DE); Michael Wiets, Langensendelbach (DE)

(21) Appl. No.: 17/216,606

(22) Filed: Mar. 29, 2021

(30) Foreign Application Priority Data
Mar. 31, 2020 (DE) 10 2020 204 155.5

(51) Int. Cl. A61B 17/22; A61B 34/30; A61M 25/09; A61B 34/20; A61B 5/00; A61B 5/02

(52) U.S. Cl. CPC A61B 17/22; A61B 34/30; A61M 25/09041; A61B 34/20; A61M 2025/0042; A61B 5/02007; A61B 5/4836; A61B 2034/301; A61B 5/6851

(57) ABSTRACT

For improved treatment of occlusions, a microcatheter guidewire unit for use in a hollow organ including a catheter body with a distal end, a proximal end, and at least two guidewires with guidewire tips is provided. The first guidewire and/or a tip of the first guidewire has a higher rigidity than a second guidewire and/or a tip of the second guidewire. The first guidewire and the second guidewire are passed through the catheter body and arranged such that the tip of the first guidewire and the tip of the second guidewire may be advanced or retracted independently of one another along longitudinal axes.

This application claims the benefit of German Patent Application No. DE 10 2020 204 155.5, filed Mar. 31, 2020, which is hereby incorporated by reference in its entirety.

### BACKGROUND

The present embodiments relate to a microcatheter guidewire unit for use in a hollow organ, a robotic catheter system, and a medical system.

A chronic total occlusion (CTO) designates the occlusion of an arterial blood vessel that impedes blood flow beyond the blockage. This may result in muscles and also vital organs being undersupplied. A CTO may occur in both coronary and peripheral arteries and may lead to severe disease and even death. The cause of a CTO is generally atherosclerosis.

To reverse the effects of a CTO, it is necessary to restore an adequate blood flow. This may be achieved in two ways: either the CTO is recanalized or the blood flow is restored at the distal end of the CTO via a bypass, if anatomically possible. With recanalization, in a first act, the CTO is passed with the aid of a medical device, such as, for example, a guidewire. FIG. 1 shows a blood vessel 1 with an occlusion 2, where a catheter 3 (e.g., a microcatheter) with a guidewire 4 is inserted into the blood vessel. CTOs may consist of soft or hard plaque. Soft plaque may usually be passed easily with the aid of special CTO guidewires. Then, a balloon catheter may be pushed in behind in order to dilate the constriction and the enlarged, ideally healthy, vessel diameter may possibly be additionally stabilized with a stent. However, if the plaque has been present in the vessel for several months or years, the plaque calcifies and becomes fibrotic so that passing the CTO with a guidewire without damaging or puncturing the vessel wall is virtually impossible or extremely time-consuming. Even experienced interventionalists or cardiologists need several hours to overcome a calcified CTO through any microchannels that may be present or to drill a hole through the calcified CTO. In the case of a very hard CTO, generally, a particularly rigid guidewire is used, but this does not have the flexibility required to overcome microchannels. If it is completely impossible to drill a hole in the CTO, a bypass is necessary, but this entails higher rates of complication and higher costs.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an apparatus that enables an interventionalist to drill through a CTO quickly and with the lowest possible risk to the blood vessel is provided.

A microcatheter guidewire unit according to the present embodiments for use in a hollow organ includes a catheter body with a distal end and a proximal end and at least two guidewires with guidewire tips. A first guidewire of the at least two guidewires and/or a tip of the first guidewire has a higher rigidity than a second guidewire of the at least two guidewires and/or a tip of the second guidewire. The first guidewire and the second guidewire are passed through the catheter body and are arranged such that the tip of the first guidewire and the tip of the second guidewire may be advanced and/or retracted (e.g., additionally rotated) independently of one another along corresponding longitudinal axes, respectively. A microcatheter guidewire unit of this kind with two guidewires of different rigidity may easily be used simultaneously to probe toward an occlusion (e.g., a CTO) (e.g., through microchannels with the less rigid guidewire) and to drill into the occlusion (e.g., with the more rigid guidewire). This makes it possible to probe or drill a route through the occlusion without replacing the apparatus and hence much more quickly. The independent mobility of the two guidewires has the advantage that the guidewires may be used alternately as required and do not impede one other when the respective other one may be retracted. However, the two guidewires are at least partially passed together through the catheter body and may thus also be navigated together to a site of use in a cavernous organ.

According to one embodiment, the catheter body includes at least two channels or two lumens, and each guidewire is arranged at least partially individually in a respective one of the channels or lumens. This provides that the two guidewires are unable to get caught in one another, do not impede one other, do not create friction against one another, and thus may always be used reliably independently of one another.

According to a further embodiment, the rigidities of the two guidewires and/or tips differ by a factor of at least 1.5 or by a factor of 2.0. This provides that the two guidewires may be used for different purposes (e.g., probing for the less rigid guidewire and drilling for the more rigid guidewire). The rigidity of guidewires may, for example, be established by different thicknesses, material properties (e.g., hardness) or the structure (e.g., core material, coating).

According to a further embodiment, the microcatheter guidewire unit includes at least one further guidewire (e.g., a third guidewire). It is also possible for a plurality of guidewires to be provided. These may all have different properties (e.g., rigidities) or resemble one of the two first guidewires in a redundant manner.

The robotic catheter system according to the present embodiments with the microcatheter guidewire unit includes at least one control unit and one robot-assisted drive system with a drive and a drive mechanism. The drive system (e.g., in the region of the proximal end of the two guidewires) is detachably coupled to at least one of the guidewires. The drive system is configured to automatically or semi-automatically advance and retract the at least one of the two guidewires in an axial direction independently of the other guidewire and/or also additionally to move the at least one guidewire rotationally.

In principle, robotic catheter systems are known, for example, from EP 3406291 B1, by which an automatic or semiautomatic advance of an object (e.g., a catheter and/or guidewire) in a cavernous organ of a patient may be effected. Coupling a robotic catheter system of this kind to a microcatheter guidewire unit as described above and embodying the robotic catheter system to move the microcatheter guidewire unit enables the advantages of the microcatheter guidewire unit according to the present embodiments to be potentiated. While the robot-assisted drive system and the control unit are arranged outside the body of the patient, the microcatheter guidewire unit is, for example, inserted via an introducer sheath at an entry point into the hollow organ of the patient and is at least partially located therein in the event of an examination or treatment. A robotic catheter system according to the present embodiments enables occlusions in the cavernous organ of the patient to be treated quickly and with particularly low risk to the patient.

The robot-assisted drive system is, for example, coupled to the first more rigid guidewire (e.g., in that the proximal end of this guidewire partially engages with the drive mechanism). This may, for example, be achieved in that the drive system includes a cassette through which the guidewire is passed and motion-coupled to one or more actuators of the drive mechanism. This may effect an advance or retraction movement and a rotational movement of the guidewire in the hollow organ. The movements are actuated by the control unit. This may be operated by a user (e.g., by remote manipulation), for example, via an operating unit, such as, for example, an input field, a keyboard, or a lever. It is also possible for a fully automatic control system to be present, which, for example, uses a previously planned path or is automatically actuated and regulated based on parameters.

In one embodiment, the catheter body may also be detachably coupled to the robot-assisted drive system, where the one or a further drive system has the effect that, when actuated automatically or semi-automatically, the catheter body may be advanced and retracted in an axial direction (e.g., also moved rotationally). Herein, semi-automatic actuation may also, for example, be actuation that may be transmitted to the control unit by an operator via an input unit (e.g., joystick, touchpad, etc.).

According to a further embodiment, the robotic catheter system includes a first robot-assisted drive system and a second robot-assisted drive system with at least one drive and a drive mechanism. Alternatively, the robotic catheter system includes a first robot-assisted drive system with two drives and two drive mechanisms, where each guidewire is detachably coupled with one of the robot-assisted drive systems or one of the drive mechanisms and where the drive systems or the drive mechanisms are configured to automatically or semi-automatically advance and retract the two guidewires independently of one another in an axial direction and, optionally, to move the two guidewires rotationally. Herein, the two drive systems may form a structural unit or be arranged separately from one another. In the event of a drive system with two drive mechanisms and drives, these form a structural unit. The guidewires are coupled to the respective drive systems or drive mechanisms (e.g., in that the guidewires partially engage with the respective drive mechanism). This may, for example, be achieved in that the two drive systems each include a cassette through which the respective guidewire is passed and which is motion-coupled to one or more actuators of the drive mechanism. However, it is also possible for two drive mechanisms to be arranged in a cassette. Overall, an advance or retraction movement and also a rotation movement of the guidewire may be effected in the hollow organ. In this way, both guidewires may be actuated semi- or fully automatically and moved individually as required.

In the case of a plurality of guidewires being present (e.g., three or more), it is also possible for a plurality of robot-assisted drive systems to be present or one robot-assisted drive system with a plurality of drives and drive mechanisms to be present.

According to a further embodiment, the first robot-assisted drive system or the first drive mechanism is coupled to the first guidewire and configured to drive (e.g., automatically) the first guidewire (e.g., the more rigid guidewire) such that the first guidewire executes a plurality of short-stroke alternating advance and retraction movements in rapid succession. As a result of this, the advance movements may exert a force on an object (e.g., an occlusion) in the hollow organ. In this way, the more rigid guidewire acts like a hammer drill or chisel, which strikes the occlusion (e.g., hardened occlusion), and in this way, may drill a path into the occlusion. The speed and the stroke of the respective movement may be adapted to the individual rigidity of the guidewire and hardness of the occlusion. For example, a frequency of, for example, 1 Hz to 1000 Hz and a stroke of, for example, 0.1 mm to 1 mm may be used.

According to a further embodiment, the first robotic drive system is coupled to the first guidewire, and the second drive system is coupled to the second guidewire. The robotic catheter system is configured to advance the second guidewire within a hollow organ as far as an occlusion, then to advance the first guidewire as far as the occlusion and to cause a plurality of short-stroke alternating advance and retraction movements to be executed in rapid succession. As a result of this, a force is exerted on the occlusion. The advantage of such a workflow consists in the fact that, for example, in softer occlusions or microchannels, the less rigid guidewire may first probe out a route. Here, the less rigid guidewire acts as a sort of “pathfinder wire”. Herein, the less rigid guidewire may be advanced manually, automatically, or semi-automatically; it is possible, for example, for imaging to be provided in order to check the position. If the less rigid guidewire reaches its limits due to hardening or the lack of microchannel, the more rigid guidewire is used, which then functions as a hammer drill and drills through the harder part of the occlusion. The detection of the hardened constriction may, for example, be determined by torque measurement or optically from the imaging. Then, the more rigid guidewire may be pushed as far as the constriction, and optionally, the less rigid guidewire may be retracted before the rapid strokes are executed. Then, for example, after a certain number of strokes or a certain period of time or distance, the constriction is checked. Such a check may also be performed live, for example, by imaging. Then, depending upon the result, either the non-rigid guidewire is reused, or the procedure is continued with the rigid guidewire.

The medical system according to the present embodiments includes a robotic catheter system and a medical imaging device (e.g., an X-ray device, a computed tomography X-ray device, a magnetic resonance tomography device, an ultrasound device, etc.). The imaging device is, for example, used to monitor the progress of an intervention (e.g., to remove an occlusion).

According to one embodiment, the medical system also includes a sensor for detecting an occlusion in the hollow organ. For example, the sensor is formed by a torque sensor, which is, for example, arranged on the second robotic drive system, and an occlusion is detected based on the advancing behavior of the second guidewire. Alternatively, an occlusion may also be detected by imaging (e.g., using the imaging device or by another method).

### BRIEF DESCRIPTION OF THE DRAWINGS

FIG. 1 is a view of a blood vessel of a patient with an occlusion and an inserted known catheter guidewire unit;

FIG. 2 is a view of a microcatheter guidewire unit according to an embodiment with two guidewires with different properties;

FIG. 3 shows a medical system with one embodiment of a robotic catheter system with a drive system;

FIG. 4 is a view of a further robotic catheter system with two drive systems arranged separately;

FIG. 5 is a view of a drive system coupled to an individual guidewire;

FIG. 6 is a view of a drive system that may be coupled to two guidewires; and

FIG. 7 shows a sequence of acts of one embodiment of a method for use with a robotic catheter system.

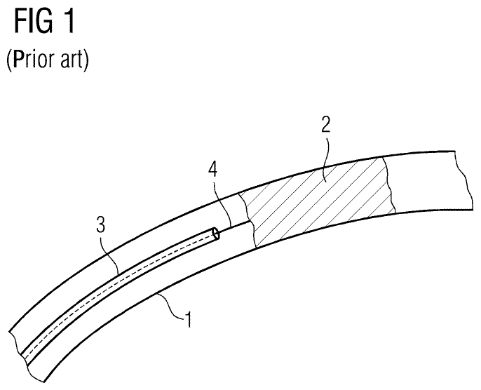

### DETAILED DESCRIPTION

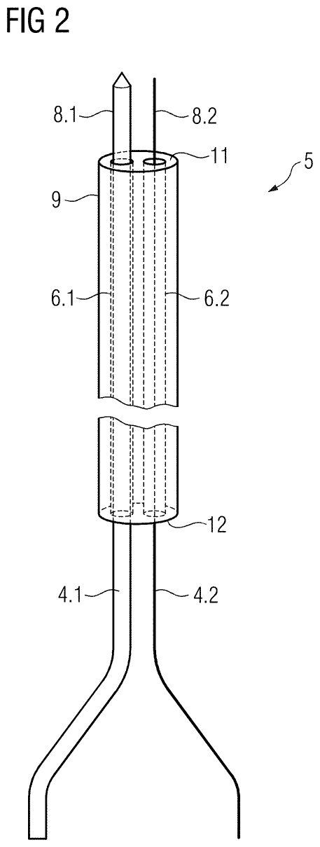

FIG. 2 shows a microcatheter guidewire unit 5 for use in a hollow organ (e.g., a vessel or vascular system) of a patient. The microcatheter guidewire unit 5 includes a catheter body 9 with a distal end 11 and a proximal end 12 and at least two guidewires 4.1, 4.2 with guidewire tips 8.1; 8.2. In one embodiment, a first guidewire 4.1 and a second guidewire 4.2 are passed through the catheter body 9. The first guidewire 4.1 is arranged in a first channel 6.1, and the second guidewire 4.2 is arranged in a second channel 6.2. The first guidewire-tip 8.1 and the second guidewire-tip 8.2 may be advanced beyond the distal end 11 of the catheter body 9 and retracted again. The first guidewire 4.1 and the second guidewire 4.2 are passed through the channels and arranged such that the guidewires or guidewire tips may be advanced and retracted again independently of one another along corresponding longitudinal axes. At the same time, the guidewires may also be additionally moved rotationally independently of one another, which provides navigation through curvatures in hollow organs. The entire microcatheter guidewire unit 5 may also be moved translationally and/or rotationally forward in order to be navigated into a hollow organ.

Herein, the first guidewire 4.1 and/or the first guidewire-tip 8.1 of the first guidewire 4.1 have a higher rigidity than the second guidewire 4.2 and/or the second guidewire-tip 8.2 of the second guidewire. Thus, it may be advantageous for the rigidities of the two guidewires and/or guidewire tips to differ by a factor of at least 1.5 or 2.0. This provides that the two guidewires may be used particularly efficiently for different purposes (e.g., probing for the less rigid guidewire and drilling for the more rigid guidewire).

The rigidity of guidewires may, for example, be established by different thicknesses, material properties (e.g., hardness) or the structure (e.g., core material, coating). The thickness of the guidewires may be, but does not have to be, different, and the sharpness of the guidewires may be, but does not have to be, different. With guidewires, the flexural rigidity, for example, is relevant. This is made up of the modulus of elasticity and the area moment of inertia. All standard commercially available guidewires may be used as guidewires, such as, for example, a Pilot 50, an Asahi Gaia Second, or an Asahi Confianza as a hard wire. The usual thickness of guidewires is about 0.26-0.36 mm. The guidewires may also be thicker or thinner. Some guidewires may be made hydrophilic by pre-treatment or coating; a known coating is, for example, Teflon.

It is also possible for further guidewires to be arranged in the microcatheter guidewire unit 5 (e.g., in further channels) and likewise to be independently movable. Such a further guidewire may, for example, have a more strongly curved guidewire-tip, which is embodied to overcome strong curvatures (e.g., bifurcations, ostia, left atrium, etc.), for example.

If the microcatheter guidewire unit 5 is introduced into a hollow organ (e.g., a vascular system of a patient), the microcatheter guidewire unit 5 may easily be used at the same time to probe toward an occlusion (e.g., a CTO) through, for example, microchannels with the less rigid guidewire and to drill into the occlusion (e.g., with the more rigid guidewire). This makes it possible to probe or drill a route through the occlusion without replacing the apparatus and hence much more quickly. Herein, the second less rigid guidewire 4.2 is the “pathfinder wire”, and the first rigid guidewire 4.1 is the “hammer drill”. The two guidewires 4.1, 4.2 may be moved against one another. Thus, during an intervention, the second less rigid guidewire may first be used to probe out a microchannel in the occlusion (e.g., CTO or other constriction). If no further progress is possible, the first guidewire is pushed up to contact, the second guidewire is retracted, and then, the first more rigid guidewire is pushed forward and back with minimal impact and retraction movements in order to penetrate the occlusion. This may, for example, be performed manually.

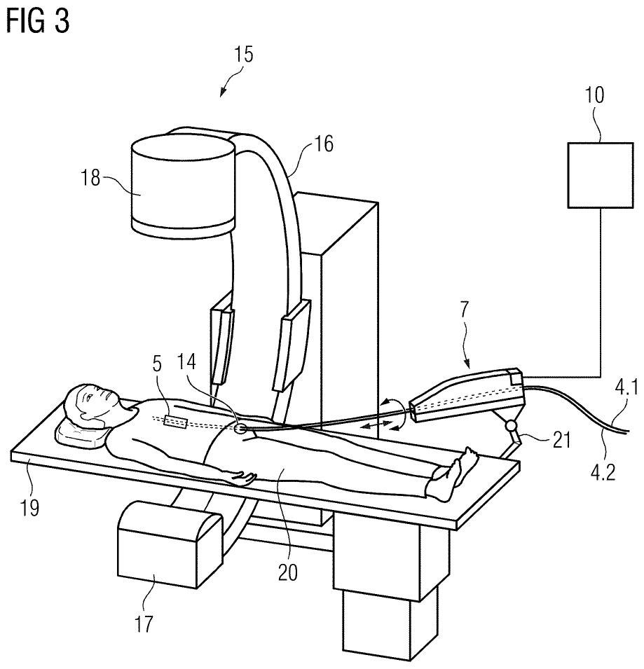

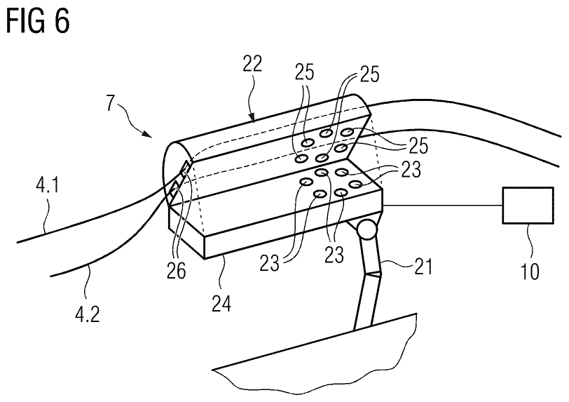

The following describes an apparatus for the semi-automatic or automatic advance of the guidewires. Herein, semi-automatic actuation may also, for example, be actuation that may be transmitted to the control unit by an operator via an input unit (e.g., joystick, touchpad, etc.). FIG. 3 shows one embodiment of a robotic catheter system with a microcatheter guidewire unit 5, at least one robot-assisted drive system 7, and a control unit 10. The robot-assisted drive system 7 has at least one drive and at least one drive mechanism, shown in more detail in FIGS. 5 and 6, and is detachably coupled to at least one of the guidewires 4.1, 4.2. The drive system 7 is also embodied to automatically or semi-automatically advance or retract the at least one guidewire independently of the other guidewire in an axial direction and, for example, also to move the at least one guidewire rotationally. In principle, robotic catheter systems are known by which a (semi)automatic advance of a (micro)catheter and/or guidewire in a cavernous organ of a patient may be effected (e.g., such as those made by the company Corindus Inc.; see EP 3406291 B1).

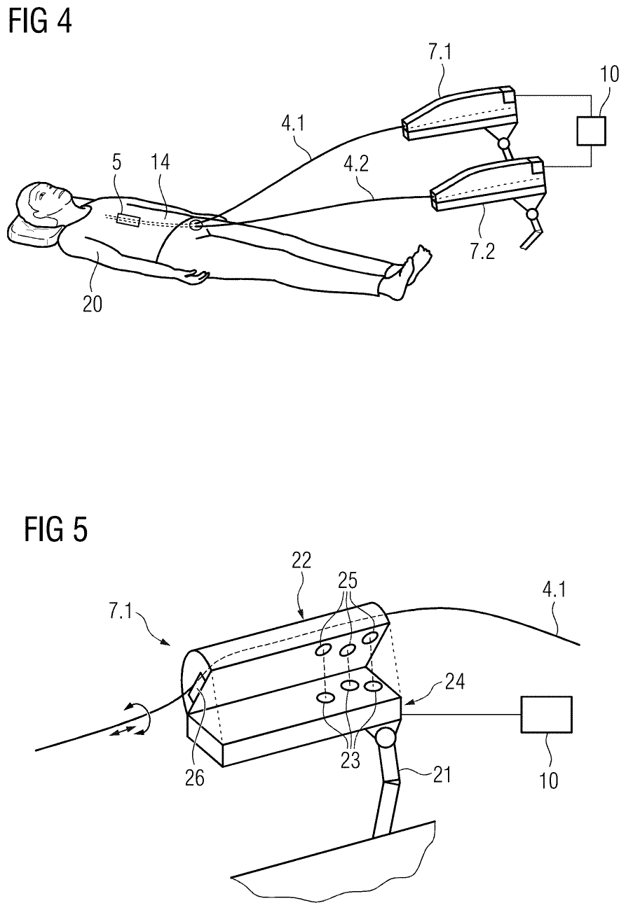

The present robotic catheter system may, for example, automatically or semi-automatically advance and retract both guidewires 4.1 and 4.2 independently of one another in an axial direction and may also move both guidewires 4.1 and 4.2 rotationally. For this purpose, either drive system 7 includes at least two drive mechanisms and at least two drives, where, in each case, a guidewire is detachably coupled to at least one drive mechanism; alternatively, at least two drive systems 7.1 and 7.2 are provided, where each includes at least one drive mechanism, and a drive and is detachably coupled to a respective guidewire (see FIG. 4).

FIG. 5 shows a first drive system 7.1 that is coupled to an individual guidewire (e.g., the first guidewire 4.1). The drive system includes a drive system base element 24 and a cassette element 22 (e.g., a replaceable cassette element). Further, the drive system base element 24 may include at least one (e.g., three) actuator elements 23 (e.g., an electric motor), where the control unit 10 is configured to control the at least one actuator element 23. The cassette element 22 may be coupled (e.g., mechanically and/or electromagnetically and/or pneumatically) to the drive system base element 24 and, for example, to the at least one actuator element 23. Herein, the cassette element 24 may include at least one transmission element 25 that may be moved through the coupling between the cassette element 22 and the drive system base element 24. In this way, the at least one transmission element 25 may be motion-coupled to the at least one actuator element 23. Hence, the transmission element 25 is configured to transmit a movement of the actuator element 23 to the first guidewire 4.1 such that the first guidewire 4.1 is moved in a translatory manner and/or rotated about the longitudinal extension direction. The at least one transmission element 25 may, for example, include a roller and/or roll and/or diaphragm. A second drive system 7.2 may have a same design to that of the first drive system 7.1 and be coupled to the second guidewire 4.2 for the movement thereof. Both drive systems 7.1 and 7.2 may be actuated via the control unit, where the regulation may take place separately.

In the case of a single drive system 7 with two drive mechanisms and drives, these form a structural unit (see FIG. 6). The drive system base element 24 includes at least two (e.g., six) actuator elements 23, and the control unit 10 is configured to control the at least two actuator elements 23. The cassette element 22 may be coupled to the drive system base element 24 and, for example, the at least two actuator elements 23, in that the cassette element 24 includes at least two transmission elements 25 that may be moved through the coupling between the cassette element 23 and the drive system base element 24. Hence, the transmission elements 25 are configured to transmit a movement of the actuator elements 23 to the two guidewires 4.1 and 4.2, such that the two guidewires 4.1 and 4.2 are moved independently of one another in a translatory manner and/or rotated about the longitudinal extension direction.

The drive system or drive systems 7, 7.1, 7.2 may be fastened to a fastening element 21 (e.g., a stand and/or robot arm) and attached by this to, for example, a patient bench 19.

FIG. 3 also shows a patient 20 on the patient bench 19 on which, for example, an interventional procedure for the recanalization of an occlusion (e.g., CTO) may be performed. For such a procedure, the microcatheter guidewire unit 5 may be inserted via an introducer sheath at an entry point 14 into the hollow organ (e.g., the vascular system) of the patient 20.

A medical imaging device (e.g., an X-ray device 15) with a C-arm 16 on which an X-ray source 18 and an X-ray detector 17 are arranged may also be provided and, together with the robotic catheter system, form a medical system according to the present embodiments.

The control unit 10 of the robotic catheter system may be configured to actuate the movements of the guidewires automatically and/or semi-automatically. In the case of semi-automatic actuation, the control unit may, for example, be connected to one or more input units, via which a user's control commands may be transmitted and then used by the control unit to actuate the drive systems. A fully automatic control also enables previously planned paths or movement profiles to be implemented. For example, a user may perform path planning in accordance with known methods in a 3D volume (e.g., CT or MR) in advance. It is also possible for an additional regulation unit that actuates the guidewires and regulates movements based on sensor data (e.g., via imaging, the resistance of the occlusion, torques) and parameters to be provided. It may also be possible to switch between two functionalities (e.g., automatic and/or semi-automatic).

In one embodiment, a first robot-assisted drive system or the first drive mechanism is coupled to the first guidewire and configured to drive the first guidewire, for example, automatically such that the first guidewire executes a plurality of short-stroke alternating advance and retraction movements in rapid succession. This may be actuated via the control unit. This function may thus be activated by a user or automatically when required. As a result, the advance movements exert a force on an object (e.g., an occlusion) in the hollow organ. In this way, the more rigid guidewire acts like a hammer drill or chisel that strikes the occlusion (e.g., hardened occlusion) and, in this way, is able to drill a path into the occlusion. The speed and the stroke of the respective movement may be adapted to the individual rigidity of the guidewire and hardness of the occlusion. For example, a frequency of 1 Hz to 1000 Hz and a stroke of, for example, 0.1 mm to 1 mm may be used.

The robotic catheter system may be configured to advance the second guidewire within a hollow organ as far as the occlusion, then to advance the first guidewire as far as the occlusion and cause a plurality of short-stroke alternating advance and retraction movements to be executed there in rapid succession; as a result of this, a force is exerted on the occlusion.

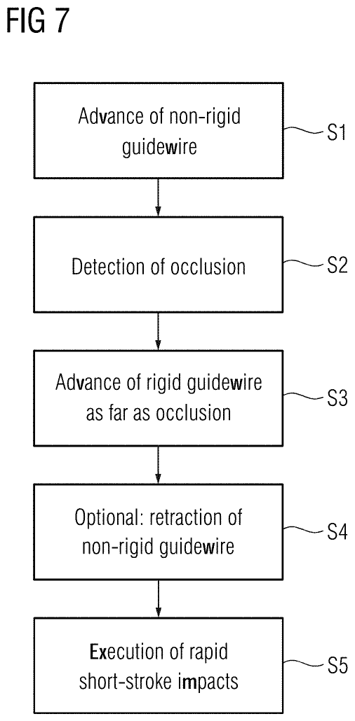

FIG. 7 describes a method that may be carried out semi-automatically or automatically by the robotic catheter system or the medical system. For the method, the microcatheter guidewire unit 5 has already been inserted in the hollow organ (e.g., the vascular system) of the patient 20, and, for example, with the aid of imaging, the guidewire tips are positioned as perpendicular as possible to the obstruction or, if the hollow organ is curved, to target the distal end of the occlusion. In a first act S1, the second less rigid guidewire is advanced in the direction of the occlusion or in the region of the occlusion through microchannels. This may, for example, be supported by imaging. In a second act S2, an occlusion that cannot be passed by the non-rigid guidewire is detected. This may, for example, also be performed by imaging or also by a torque sensor 26 positioned, for example, on the drive system or the catheter system (e.g., the guidewire tip). In a third act S3, the first more rigid guidewire is advanced as far as the contact (e.g., likewise performed by a torque sensor, by imaging or distance measurement). Then, optionally, the second guidewire may be retracted in a fourth act S4. Then, in a fifth act S5, the first guidewire a executes a plurality of short-stroke alternating advance and retraction movements in rapid succession in order to drill through the occlusion. The stroke may, for example, be defined by a user, or the required size of the respective next stroke may be determined by a pre-trained machine-learning algorithm based on image information from the imaging, the behavior of the guidewire with previous strokes, or the values of the torque sensor. It is also possible to infer the histology of the occlusion based on information from imaging, the sensors, or the course of the previous method, and for impact depth and energy to be determined on the basis thereof.

The robotic catheter system may, for example, be configured for the fully automatic performance of the method based on a planned path. The robotic catheter system may be configured to advance the second guidewire within a hollow organ as far as the occlusion, then to advance the first guidewire as far as the occlusion, and to cause a plurality of short-stroke alternating advance and retraction movements to be executed in rapid succession there. As a result of this, a force is exerted on the occlusion. The robotic catheter system may also be configured, in dependence on the measured resistance of the occlusion, to use the respective appropriate guidewire (e.g., the second less rigid guidewire when measuring a soft resistance and the first rigid guidewire when measuring a hard resistance).

The present embodiments may be briefly summarized as follows: for improved treatment of occlusions (e.g., CTOs) in cavernous organs, a microcatheter guidewire unit for use in a hollow organ is provided. The microcatheter guidewire unit includes a catheter body with a distal end and a proximal end, and at least two guidewires with guidewire tips. The first guidewire and/or the tip of the first guidewire have a higher rigidity than the second guidewire and/or the tip of the second guidewire. The first guidewire and the second guidewire are passed through the catheter body and arranged such that the tip of the first guidewire and the tip of the second guidewire may be advanced and/or retracted independently of one another along longitudinal axes and/or be rotated. Also provided is a robotic catheter system with a microcatheter guidewire unit including at least one control unit and one robot-assisted drive system with a drive and a drive mechanism. The drive system (e.g., in the region of the proximal end of the two guidewires) is detachably coupled to at least one of the guidewires. The drive system is configured to automatically or semi-automatically advance and retract the at least one of the two guidewires independently of the other guidewire in the axial direction and to rotate the at least one guidewire.

The exemplary embodiments were selected and described in order to be able to describe the principles on which the invention is based and possible applications in practice in the best possible manner. As a result, experts may modify and use the invention and various exemplary embodiments of the invention in an optimum manner with respect to the intended purpose.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

1. A microcatheter guidewire unit for use in a hollow organ, the microcatheter guidewire unit comprising:
a catheter body with a distal end and a proximal end; and
at least two guidewires with guidewire tips,
wherein a first guidewire of the at least two guidewire, a tip of the first guidewire, or the first guidewire and the tip of the first guidewire have a higher rigidity than a second guidewire of the at least two guidewires, a tip of the second guidewire, or the second guidewire and the tip of the second guidewire, and
wherein the first guidewire and the second guidewire are passed through the catheter body and are arranged such that the tip of the first guidewire and the tip of the second guidewire are advanceable or retractable independently of one another along corresponding longitudinal axes.

2. The microcatheter guidewire unit of claim 1, wherein the catheter body comprises at least two channels or two lumens, and each guidewire of the at least two guidewires is arranged at least partially individually in a respective one of the at least two channels or two lumens.

3. The microcatheter guidewire unit of claim 1, wherein rigidities of the first guidewire and the second guidewire, the tip of the first guidewire and the tip of the second guidewire, or the first guidewire and the second guidewire and the tip of the first guidewire and the tip of the second guidewire, respectively, differ by a factor of at least 1.5.

4. The microcatheter guidewire unit of claim 3, wherein the rigidities of the first guidewire and the second guidewire, the tip of the first guidewire and the tip of the second guidewire, or the first guidewire and the second guidewire and the tip of the first guidewire and the tip of the second guidewire, respectively, differ by a factor of at least 2.

5. The microcatheter guidewire unit of claim 1, further comprising at least one further guidewire.

6. A robotic catheter system comprising:
a microcatheter guidewire unit for use in a hollow organ, the microcatheter guidewire unit comprising:
a catheter body with a distal end and a proximal end; and
at least two guidewires with guidewire tips, wherein a first guidewire of the at least two guidewire, a tip of the first guidewire, or the first guidewire and the tip of the first guidewire have a higher rigidity than a second guidewire of the at least two guidewires, a tip of the second guidewire, or the second guidewire and the tip of the second guidewire, and wherein the first guidewire and the second guidewire are passed through the catheter body and are arranged such that the tip of the first guidewire and the tip of the second guidewire are advanceable or retractable independently of one another along corresponding longitudinal axes;
at least one control unit; and
a robot-assisted drive system with a drive and a drive mechanism,
wherein the robot-assisted drive system is detachably coupled to one or more of the at least two guidewires, and
wherein the robot-assisted drive system is configured to automatically or semi-automatically advance and retract the one or more guidewires in an axial direction independently of other guidewires of the at least two guidewires.

7. The robotic catheter system of claim 6, wherein the drive system is detachably coupled to the one or more guidewires in a region of a proximal end of the at least two guidewires.

8. The robotic catheter system of claim 6, wherein the catheter body is detachably coupleable to the robot-assisted drive system, and
wherein the robot-assisted drive system is configured such that, when actuated automatically or semi-automatically, the catheter body is advanceable and retractable in an axial direction.

9. The robotic catheter system of claim 6, wherein the robot-assisted drive system is a first robot-assisted drive system,
wherein the robotic catheter system further comprises a second robot-assisted drive system with at least one drive and at least one drive mechanism, or the first robot-assisted drive system has two drives and two drive mechanisms,
wherein each guidewire of the at least two guidewires is detachably coupled to the first robot-assisted drive system or the second robot-assisted drive system, or one of the drive mechanisms, and
wherein the first robot-assisted drive system and the second robot-assisted drive system or the drive mechanisms are configured to automatically or semi-automatically advance and retract the at least two guidewires independently of one another in an axial direction.

10. The robotic catheter system of claim 9, wherein the first robot-assisted drive system or one of the drive mechanisms is coupled to the first guidewire, and the second robot-assisted drive system or one of the drive mechanisms is coupled to the second guidewire, and
wherein the robotic catheter system is configured to advance the second guidewire within the hollow organ as far as an occlusion, then to advance the first guidewire as far as the occlusion and to cause a plurality of short-stroke alternating advance and retraction movements to be executed in rapid succession so that a force is exerted on the occlusion.

11. The robotic catheter system of claim 6, wherein the robot-assisted drive system or the drive mechanism is coupled to the first guidewire and is configured to drive the first guidewire, such that the first guidewire executes a plurality of short-stroke alternating advance and retraction movements in succession so that a force is exertable by the advance movements on an object in the hollow organ.

12. A medical system comprising:
a robotic catheter system comprising:
a microcatheter guidewire unit for use in a hollow organ, the microcatheter guidewire unit comprising:
a catheter body with a distal end and a proximal end; and
at least two guidewires with guidewire tips, wherein a first guidewire of the at least two guidewire, a tip of the first guidewire, or the first guidewire and the tip of the first guidewire have a higher rigidity than a second guidewire of the at least two guidewires, a tip of the second guidewire, or the second guidewire and the tip of the second guidewire, and wherein the first guidewire and the second guidewire are passed through the catheter body and are arranged such that the tip of the first guidewire and the tip of the second guidewire are advanceable or retractable independently of one another along corresponding longitudinal axes;
at least one control unit; and
a robot-assisted drive system with a drive and a drive mechanism, wherein the robot-assisted drive system is detachably coupled to one or more of the at least two guidewires, and wherein the robot-assisted drive system is configured to automatically or semi-automatically advance and retract the one or more guidewires in an axial direction independently of other guidewires of the at least two guidewires; and
a medical imaging device.

13. The medical system of claim 12, further comprising a sensor configured to detect an occlusion in the hollow organ,
wherein the robot-assisted drive system or one of the drive mechanism is coupled to the first guidewire, and a second robot-assisted drive system or another drive mechanism is coupled to the second guidewire, and
wherein the robotic catheter system is configured to advance the second guidewire within the hollow organ as far as the occlusion, then to advance the first guidewire as far as the occlusion and to cause a plurality of short-stroke alternating advance and retraction movements to be executed in rapid succession so that a force is exerted on the occlusion.

14. The medical system of claim 13, wherein the sensor is formed by a torque sensor arranged on the robot-assisted drive system or the robotic catheter system and is configured to detect the occlusion based on an advancing behavior of the second guidewire.

15. The medical system of claim 14, wherein the medical system is configured to execute a pre-trained machine-learning algorithm that determines a size of a respective next stroke based on imaging information of an imaging or a behavior of the first guidewire with previous strokes or values of the torque sensor.

16. The medical system of claim 12, wherein the medical imaging device is an X-ray device.

17. A method for automatic or semi-automatic advance of a microcatheter guidewire unit with two guidewires in a hollow organ, wherein a first guidewire of the two guidewires, a tip of the first guidewire, or the first guidewire and the tip of the first guidewire have a higher rigidity than a second guidewire of the two guidewires, a tip of the second guidewire, or the second guidewire and the tip of the second guidewire, the method comprising:
advancing the second guidewire within the hollow organ;
detecting an occlusion impeding the advance of the second guidewire within the hollow organ;
advancing the first guidewire as far as the occlusion;
optionally retracting the second guidewire; and
executing a plurality of short-stroke alternating advance and retraction movements of the first guidewire in rapid succession,
wherein the advance movement executes a force on the occlusion.
